(19) United States
(12) Patent Application Publication
(10) Pub. No.: US 2013/0142751 A1
(43) Pub. Date: Jun. 6, 2013

(54) Colored sodium bicarbonate (baking soda)

(71) Applicant: Patrice Pettigrew, Maple Heights, OH (US)

(72) Inventor: Patrice Pettigrew, Maple Heights, OH (US)

(21) Appl. No.: 13/373,803

(22) Filed: Dec. 1, 2011

(51) Int. Cl. A61L 9/014

(52) U.S. Cl. 424/76.1

(57) ABSTRACT

A colored, sodium bicarbonate-based, composition that is, for example, a fabric freshener and deodorizer that can be colored with non-toxic ingredients is provided. A colored baking soda composition may be used to suppress malodor, for freshening fabrics comprising sodium bicarbonate, a coloring agent and a method of use thereof comprising applying the composition uniformly to the fabric and thereafter blending into fabric to remove unwanted odor. The colored baking soda is produced by mixing same together then, in one step, the mixture of the two components undergoes simultaneous mixing-refining until the desired color is obtained. The quantities of colors will vary, the amount of coloring matter used depends upon the depth of shade it is desired to produce, colors can be toned with additional baking soda. A baking soda-based product used to remove malodor, that can be colored with non-toxic ingredients is provided.

### CROSS-REFERENCE TO RELATED APPLICATIONS

This application is the non-provisional application of provisional application 61/344,965, filed Nov. 30, 2010.

### BACKGROUND

1. Field of Invention

This invention relates to colored baking soda, which is used for freshening, deodorizing fabrics.

2. Description of Prior Art

Many fabrics installed in the home or office, such as wall to wall carpeting, stretched fabric on ceiling or walls, upholstery, drapes, vehicle floor, seats, clothes, shoes and the like, absorb cooking odors, pet odors, smoke odors, and other often obnoxious or otherwise unpleasant odors. Although these fabrics may be clean from the perspective of being free of color changing dirt, the odors they absorb render them not fresh. Since they cannot be easily removed for cleaning or freshening they present a challenge for most homeowners and for many office workers.

Currently, fabric freshening products are commercially available as either water based or aerosol products that leave fabrics wet for extended periods, requiring scrubbing and vacuuming to remove visual residues. White powder products have the disadvantage of being relatively easy to visualize the areas of application. While more effective is the colored baking soda product, which is difficult to visualize on the areas to which said product has been applied allowing you to freshen the carpet and yet still entertain guests, wear clothes, deodorize drapes, shoes, boots, etc. without knowledge of the colored baking soda's existence because of the color blending, the area of application cannot easily be seen. Because the ultimate action is to absorb malodors, a not insignificant amount of time is required to absorb the odor and the fabric will always remain dry from these products.

Currently, consumers tend to overspray an area to affect the go malodor absorption but because of the time for absorption, they leave the carpet or other fabric wet to the touch for long periods.

### SUMMARY

In accordance with the present invention a composition for freshening comprising a finely mixture of a food color, baking soda.

### OBJECTS AND ADVANTAGES

Accordingly, it would be advantageous to provide a composition for freshening, deodorizing fabrics, including carpeting, clothes, upholstery and the like, which do not have the above said drawbacks of wetness, or visual appearance. Simply sprinkled, brushed, poured onto said fabrics.

### DRAWING FIGURES

Non illustrative—the method of making a color freshening composition, the compositions may be easily formed by combining all of the baking soda, food color ingredients, in a suitable dispenser.

### CONCLUSION, RAMIFICATIONS, AND SCOPE

Accordingly, the consumer will see that this invention provides a dry, non-cleaning, odor absorbing, fabric freshening product. The product can remain on said fabric surfaces to allow the user to entertain, utilize the area, wear fabric.

What is claimed is:

1. A non-cleaning composition

2. A method for freshening a fabric, comprising baking soda, food color

3. It is a ready-to-use product, which can be easily applied and which are cheap, stable and not toxic.
